(19) United States
(12) Patent Application Publication
(10) Pub. No.: US 2009/0324498 A1
(43) Pub. Date: Dec. 31, 2009

(54) METHOD OF MEASURING QUANTITY OF IN-VIVO SUBSTANCE BY USE OF COHERENT ANTI-STOKES RAMAN SCATTERED LIGHT

(75) Inventors: Takuya Kishimoto, Tokyo (JP); Michihiro Ohnishi, Kanagawa (JP); Hidetoshi Watanabe, Chiba (JP)

Correspondence Address: WOLF GREENFIELD & SACKS, P.C., 600 ATLANTIC AVENUE, BOSTON, MA, 02210-2206, US

(73) Assignee: Sony Corporation, Tokyo (JP)

(21) Appl. No.: 12/486,290

(22) Filed: Jun. 17, 2009

(30) Foreign Application Priority Data
Jun. 26, 2008 (JP) 2008-166731

(51) Int. Cl. A61K 49/00; C12Q 1/02

(52) U.S. Cl. 424/9.1; 435/29

(57) ABSTRACT

A method of measuring the quantity of an in-vivo substance includes the steps of: irradiating an in-vivo substance with two near infrared femtosecond laser beams of different frequencies; detecting coherent anti-Stokes Raman scattered light emitted from the in-vivo substance due to coincidence of the frequency difference between the two near infrared femtosecond laser beams with a natural frequency of the in-vivo substance; and determining the quantity of the in-vivo substance, based on a peak intensity of a Raman scattering spectrum obtained.

### BACKGROUND OF THE INVENTION

1. Field of the Invention

The present invention relates to a method of measuring the quantity of an in-vivo substance. More particularly, the invention relates to a method of measuring the quantity of an in-vivo substance by utilizing scattered light in CARS (Coherent Anti-Stokes Raman Scattering).

2. Description of the Related Art

Detection of in-vivo substances has been conducted by high-performance liquid chromatography (hereinafter referred to as “HPLC”), ELISA (Enzyme-Linked ImmunoSorbent Assay), and the like. In these methods, first, a part of an in-vivo tissue is taken out, a homogenate thereof is prepared, and the tissue homogenate is centrifugally separated to prepare an extract which contains the target in-vivo substance. Thereafter, in the method based on HPLC, the thus obtained extract is fractionated, and the in-vivo substance fractionated into a specified fraction is subjected to detection and quantitative measurement by use of a UV-absorbing detector. In the ELISA method, on the other hand, the in-vivo substance in the extract is subjected to detection and quantitative measurement by an antigen-antibody reaction in which an antibody specific to the in-vivo substance is used.

J. Clin. Chem. Clin. Biochem., February 1981, 19(2): 81-87 describes a method for detection and measurement of a glycosylated protein as an in-vivo substance by HPLC. Besides, Clin. Chim. Acta., November, 1989; 185(2): 157-164 describes a method for detection and measurement of a glycosylated protein by ELISA.

In these known methods based on HPLC or ELISA, the extract of the tissue or the fraction of the in-vivo substance has to be prepared, which involves dissolution (lysis) of the tissue. Therefore, it has been extremely difficult by these methods to detect an in-vivo substance in a tissue while the tissue is kept living.

In recent years, for the purpose of detecting an in-vivo substance present in a biosample such as cultured cells while the biosample is in the living state, microscopes (CARS microscopes) based on CARS have been developed (refer to Japanese Patent Laid-open No. Hei 5-288681, Japanese Patent Laid-open No. 2002-107301, and Japanese Patent Laid-open No. 2005-62155).

Here, the Raman scattering means the phenomenon in which when a substance is irradiated with incident light of a frequency ω₁, weak scattered light of a frequency ω₁+ω_{R} or ω₁−ω_{R} is emitted. In this case, ω_{R} is a frequency intrinsic of a vibration mode of the molecule under consideration, and many spectral lines arising from vibration modes of the molecule appear in the Raman scattering spectrum (hereinafter referred to simply as “spectrum”). Therefore, it is possible to detect the molecule by analyzing the spectrum.

The CARS is a kind of Raman scattering, and it means the phenomenon in which when two beams of different frequencies, pump light (frequency: ω_{P}) and Stokes light (frequency: ω_{S}), are incident on a substance, scattered light of a frequency 2ω_{P}−-ω_{S} is emitted from the substance. When ω_{P}−ω_{S} coincides with the natural frequency ω_{V} of the molecules of the substance, many vibration modes of the molecules are excited on a resonant basis, whereby a scattered light which is very intense and good in directivity can be obtained.

In CARS microscopes, the Stokes light (frequency: ω_{P}) is made to be a broadband light source and a plurality of vibration modes are excited simultaneously, whereby the CARS light is obtained as a spectrum. The spectrum in this instance varies depending on the molecule. Therefore, even when a plurality of species of molecules are coexisting at the same time, the respective molecules can be detected based on their spectra.

### SUMMARY OF THE INVENTION

In the methods of detecting an in-vivo substance using HPLC or ELISA according to the related art, it is usually necessary to prepare an extract of a tissue or a fraction of the in-vivo substance, which involves intricate operations. In these known methods, besides, it may be necessary to dissolve (lyse) the tissue, so that it is extremely difficult to detect the in-vivo substance present in a tissue while keeping the tissue alive.

Thus, there is a need for a method of measuring the quantity of an in-vivo substance by which measurement can be made by simpler operations than those in the related art and by which an in-vivo substance present in a living tissue can also be detected.

According to an embodiment of the present invention, there is provided a method of measuring the quantity of an in-vivo substance including the steps of: irradiating an in-vivo substance with two near infrared femtosecond laser beams of different frequencies; detecting coherent anti-Stokes Raman scattered light emitted from the in-vivo substance due to coincidence of the frequency difference between the two near infrared femtosecond laser beams with a natural frequency of the in-vivo substance; and determining the quantity of the in-vivo substance, based on a peak intensity of a Raman scattering spectrum obtained.

In the method of measuring the quantity of an in-vivo substance, the in-vivo substance as an object of measurement may be glycosylated hemoglobin.

In the present invention, the expression “near infrared femtosecond laser beam” refers to a laser beam which has a pulse width in the range of from femtosecond order to picosecond order (in the sub-picosecond range).

In addition, the term “in-vivo substance” refers to a chemical substance present in a living body tissue or cell. The in-vivo substance widely includes amino acids, peptides, proteins, nucleotide, nucleoside, sugars, lipid, vitamins, hormones, metallic elements, and metallic element-containing proteins.

According to the method of measuring the quantity of an in-vivo substance pertaining to the present invention, measurement of in-vivo substances in living tissues can be carried out through simple operations.

### BRIEF DESCRIPTION OF THE DRAWINGS

FIG. 1 is a schematic diagram showing an exemplary configuration of an apparatus used in the method of measuring the quantity of an in-vivo substance according to an embodiment of the present invention;

FIG. 2 is a schematic diagram showing another exemplary configuration of the apparatus used in the method of measuring the quantity of an in-vivo substance according to an embodiment of the present invention;

FIG. 3 illustrates the principle of generation of CARS;

FIGS. 4A and 4B are schematic diagrams illustrating the method of measurement of an in-vivo substance in a blood vessel in a cutaneous tissue, in which FIG. 4A illustrates a method for measuring the quantity of HbAlc in erythrocyte, and FIG. 4B illustrates a method for measurement of glycosylated albumin or the like in blood; and

FIG. 5 is a diagram showing a typical example of the spectrum of HbAlc.

### DETAILED DESCRIPTION OF THE PREFERRED EMBODIMENTS

Now, preferred embodiments for carrying out the present invention will be described below referring to the drawings. Incidentally, the embodiments described below are merely typical embodiments, and they should not be construed as limitative of the present invention.

In the method of measuring the quantity of an in-vivo substance according to an embodiment of the present invention, an in-vivo substance is irradiated (illuminated) with two near infrared femtosecond laser beams of different frequencies, CARS scattered light generated from the in-vivo substance due to coincidence of the frequency difference between the two near infrared femtosecond laser beams with the natural frequency of the in-vivo substance is detected, and the quantity of the in-vivo substance is measured based on peak intensities of the spectrum obtained.

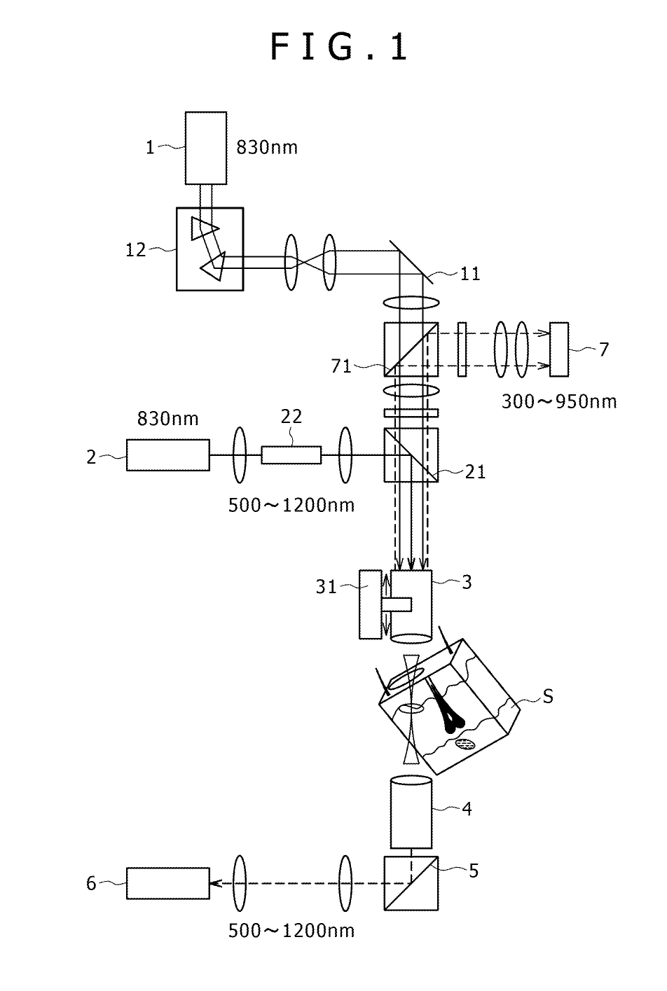

Now, the method of measuring the quantity of an in-vivo substance will be described in detail below, referring to the apparatus configuration shown in FIG. 1. The apparatus has a configuration obtained by adaptation of a related-art CARS microscope to the method of measuring the quantity of an in-vivo substance according to an embodiment of the present invention.

The apparatus shown in FIG. 1 includes: a first pulsed laser generating device 1 operable to generate pump light; a second pulsed laser generating device 2 operable to generate Stokes light; a galvanomirror 11 and a dichroic mirror 21 which are operable to combine the pump light and the Stokes light with each other on the same optical path; an objective lens 3 by which the pump light and the Stokes light thus combined are condensed to a point in a living tissue (sample) S; a condenser lens 4 operable to condense scattered light which is generated from the sample S and which contains anti-Stokes light shorter than the pump light in wavelength; and a mirror 5 and a spectroscope 6 operable to detect the scattered light thus condensed. In the figure, symbol 12 denotes a group velocity control system.

As each of the pulsed laser generating devices 1 and 2, there can be used a mode synchronized laser such as a titanium sapphire or erbium-doped fiber laser medium. In addition, the wavelengths of the near infrared femtosecond laser beams generated from the pulsed laser generating devices 1 and 2 can be appropriately selected in the range of 650 to 1100 nm. For example, at a wavelength of 830 nm, the pulse width is not more than 200 fs and the repetition frequency is 80 MHz. Besides, output stability is about ±0.5, and mean beam output is about 2 W.

In the figure, symbol 22 denotes a hollow fiber for converting the Stokes light to broadband light. With the frequency ω_{P} of the pump light fixed and with the frequency ω_{S} of the Stokes light made to be in broadband by the hollow fiber 22, the pump light and the Stokes light are condensed by the objective lens 3 to a location (focal point) in the sample S where the in-vivo substance is present. As a result, a multiple photon excitation process of the in-vivo substance is induced, and the resulting CARS scattered light is analyzed by the spectroscope 6, to obtain a spectrum.

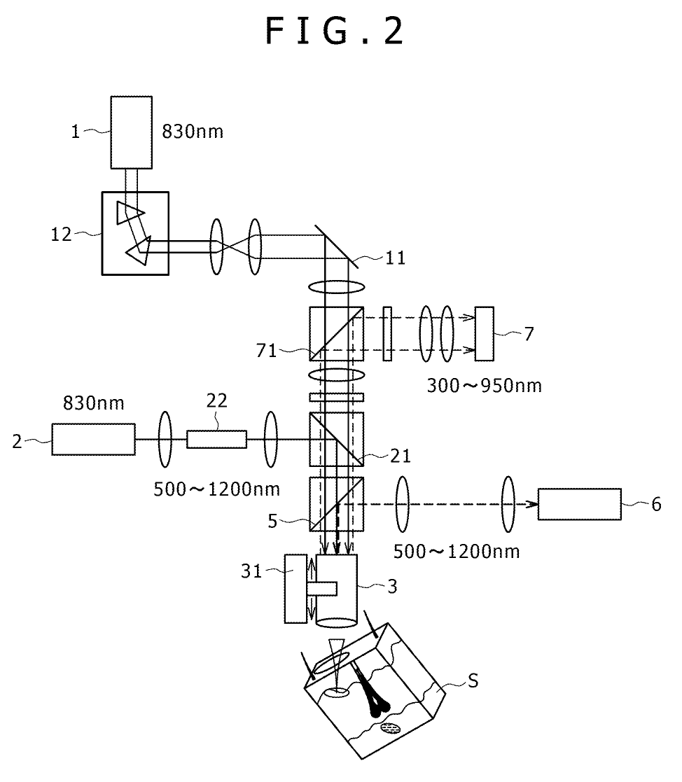

Or, as shown in FIG. 2, the CARS scattered light generated at the sample S and returning to the objective lens 3 may be condensed by the mirror (dichroic mirror) 5 to the spectroscope 6, thereby obtaining a spectrum. By this configuration, the CARS spectrum can be detected on the same side as the direction of irradiation of the sample S with the pump light and the Stokes light.

Here, the expression “multiple photon excitation” means a process in which a plurality of photons are simultaneously absorbed into a single molecule (multiple photon absorption) to effect transition to a first electron excited state or above. In this multiple photon excitation process, further, when the frequency difference between the pump light and the Stokes light coincides with a natural frequency of the in-vivo substance, CARS scattered light is generated from the in-vivo substance.

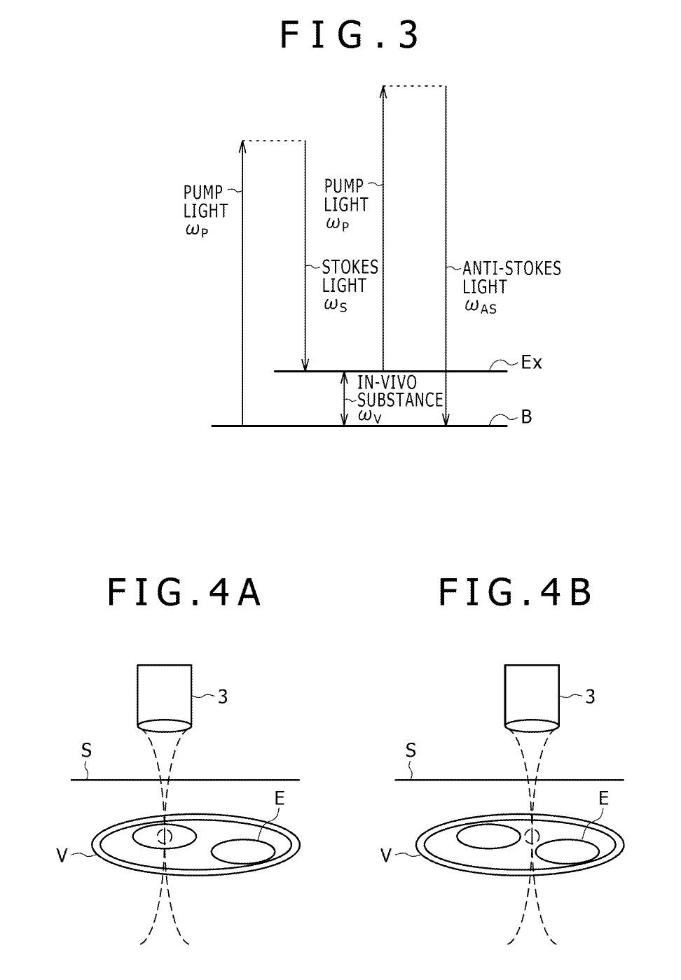

More specific description will be made based on FIG. 3. When the difference between the frequency ω_{P} of the pump light and the frequency ω_{S} of the Stokes light coincides with the natural frequency ω_{V} of an in-vivo substance in a sample S, the in-vivo substance being in a ground state B undergoes resonant vibration at the frequency ω_{V}, to be brought into an excited state Ex. Then, part of the pump light at the frequency ω_{P} undergoes Doppler modulation at the natural frequency ω_{V} of the in-vivo substance, whereby anti-Stokes light of a frequency ω_{AS} is generated. In this instance, the relation represented by the following formula (1) is established.

ω_{AS}=ω_{P}+ω_{V}=2ω_{P}−ω_{S} (1)

The CARS spectrum is specific to the molecule under consideration. Therefore, even in the case where a plurality of in-vivo substances are simultaneously present, the in-vivo substances can be identifiedly detected based on the spectra. Further, based on peak intensities of the spectrum for an in-vivo substance, the quantity of the in-vivo substance can be computed.

In the multiple photon excitation, the excitation is effected by a plurality of photons and, therefore, a laser with a longer wavelength and a lower energy than those in the single photon excitation in the related art can be used. When a long-wavelength laser which is excellent in depth reaching ability and low in energy is used, an in-vivo substance present in the depth spaced from the surface of a living tissue can be excited, and measurement can be carried out for a long period of time while suppressing damage to other portions than the focal point. Thus, in the method of measuring the quantity of an in-vivo substance according to an embodiment of the present invention, in-vivo substances in living tissues or living cells can be detected directly without controlling a tissue homogenate or cell lysate.

In addition, the multiple photon excitation process and the CARS are nonlinear optical phenomena which occur when a plurality of photons reach a molecule substantially simultaneously and which are induced only at and around the focal point of laser. Therefore, an excellent spatial resolution can be obtained in the method of measuring the quantity of an in-vivo substance according to an embodiment of the present invention.

Now, the method of measuring the quantity of an in-vivo substance according to an embodiment of the present invention will be described more in detail taking, as an example, a case where measurement of glycosylated hemoglobin (HbAlc) as an in-vivo substance is carried out.

The “glycosylated hemoglobin (HbAlc)” herein is a substance formed when a sugar (e.g., glucose) in blood is linked to erythrocyte hemoglobin by a non-enzymatic reaction. The quantity of HbAlc reflects the mean value of blood sugar level in the past one to two months, and, hence, it is an important diagnostic marker as an index to a hyperglycemic state in diabetic pathologic conditions.

FIG. 4A is a schematic illustration of a method for measurement of HbAlc in erythrocyte. In the illustration, symbol S denotes the surface of a sample (in this case, a cutaneous tissue), symbol V denotes a blood vessel, and E denotes an erythrocyte in the blood vessel V. Incidentally, FIG. 4B will be referred to later.

The pump light and the Stokes light (indicated by broken lines in the illustration) are condensed by the objective lens 3 to a point (focal point) in the erythrocyte E where the HbAlc as the object of measurement is present. In this case, the near infrared femtosecond laser beams undergo little absorption by water or blood present in the cutaneous tissue and undergo little scattering by the tissue; therefore, the laser beams exhibit a high depth reaching ability. Besides, these laser beams are low in invasiveness, so that they would not injure the skin.

The laser beams in irradiation excite especially a spindle-shaped region (the region surrounded by a dotted-line ellipse in the drawing) which is sized about 30 μm in diameter in the XY plane and about 60 μm in the Z-axis direction. The excitation occurs in the region ranging from the working distance of the objective lens 3 of 200 μm to the skin surface.

Of the objective lens 3, the position in the vertical direction in FIG. 1 (the height direction) can be controlled by the focal position control mechanism 31 (see FIG. 1). This makes it possible to adjust the depth of focus in the sample S.

The HbAlc undergoes multiple photon excitation under the actions of the pump light and the Stokes light, to emit CARS scattered light. The CARS light is either condensed by the condenser lens 4 (see FIG. 1) or condensed by the objective lens 3 (see FIG. 2), whereby a CARS spectrum is obtained at the spectroscope 6.

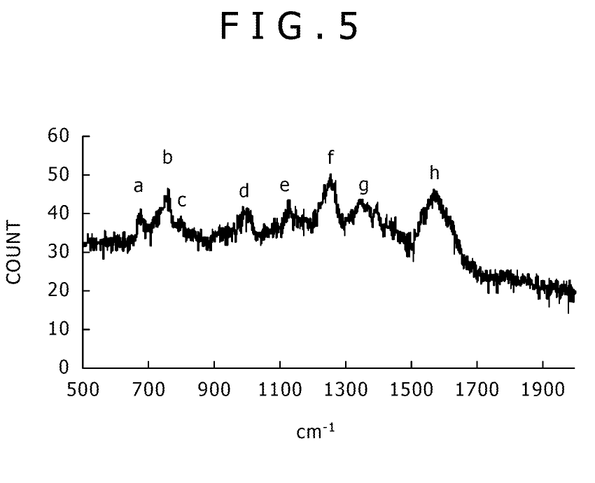

FIG. 5 shows a typical spectrum of HbAlc. In the diagram, the axis of abscissas represents wave number, and the axis of ordinates represents luminance. Acquisition of the spectrum is performed for a predetermined period of time. For instance, the spectrum is obtained for a period of 10 milliseconds from a time point of 1 millisecond after the start of irradiation with laser, and the data obtained in about 100 runs of this operation are integrated.

The spectrum region characteristic of HbAlc is observed in the range of 900 to 1700 cm⁻¹. In the diagram, the peaks denoted respectively by symbols a, b, c, d, e, f, g and h are peaks specific to the HbAlc. Particularly, the intensity of peak c depends strongly on the quantity of HbAlc, and, therefore, the quantity of HbAlc can be computed based on the intensities of the peaks.

Specifically, the local maximum peak h appearing usually at 1562 cm⁻¹ is taken as a reference, and peak intensity ratios of the peaks a, b, c, d, e, f and g to the local maximum peak h are obtained. By such a calibration with the intensity of the local maximum peak h, the quantity of HbAlc can be accurately computed while suppressing errors (dispersion) among the runs of measurement.

Besides, for known quantities of HbAlc obtained previously by quantitative determination using HPLC or ELISA, correlational expressions of peak intensity ratio and quantity of HbAlc are preliminarily obtained by the same method as above-mentioned. By use of these correlational expressions, an unknown quantity of HbAlc is computed from peak intensity ratios obtained for the unknown quantity of HbAlc.

The surface of a cutaneous tissue S as a portion to be measured is desirably a portion which is smooth, has few hairs and has a thin cuticle. In addition, the blood vessel V is preferably a venule or blood capillary near the surface of the cutaneous tissue S. For example, one of the venules or blood capillaries in the area ranging from the back of an elbow to the wrist, of a forearm, is preferably adopted as the blood vessel V. With such a portion served to measurement, the absorption and/or scattering of laser beams at the skin surface can be prevented. Further, water or an oil or the like may be applied to the skin surface to prevent the scattering of laser beams on the living body surface, whereby measurement accuracy can be enhanced more. Besides, it is desirable to shade the portion to be measured, in order to prevent penetration of environmental light into the measuring system.

In order to eliminate errors, the measurement is desirably carried out for the same erythrocyte. For this purpose, the image of the erythrocyte in a blood vessel V is obtained on a real-time basis by a CCD camera (see symbol 7 in FIG. 1) having sensitivity to infrared rays, and a CARS spectrum as to the erythrocyte E is obtained.

As shown in FIGS. 1 and 2, part of reflected light from the sample S is condensed by the objective lens 3, and is reflected by a bandpass filter 71, to be introduced to the CCD camera unit 7. The CCD camera unit 7 is connected with an image display section (not shown) so that the portion to be irradiated with the laser beams can be determined while checking the sample S on a display. This enables a process in which the image of an erythrocyte E in a blood vessel V is obtained on a real-time basis, the position of the erythrocyte E in the visual field is serially measured by processing of the obtained image, and, based on the position data, irradiation of the same portion of the same erythrocyte E with the laser beams is continued.

In the present invention, the living tissue adopted as the portion to be measured is not limited to the cutaneous tissue. For example, a nail, an ear, a fingertip, a lip, a retina, a scalp hair and the like may also be adopted, depending on the portion where the in-vivo substance as the object of measurement is present. In the case where the cutaneous tissue is adopted as the portion of measurement, due to the presence of a connective tissue specific to the cutaneous tissue, the CARS scattered light and fluorescent light are well scattered or reflected from the inside of the tissue nearer to the skin surface than the connective tissue, so that the spectrum desired can be detected efficiently. This effect is particularly conspicuous in the case where measurement of HbAlc or the like is conducted in the condition where an erythrocyte in a blood capillary present in or around the connective tissue at a subcutaneous depth of about 200 μm is contained in the same focal point.

In the present invention, the living tissue which can be adopted as the portion to be measured is not limited to a tissue exposed at the body surface; for example, in-vivo tissues of liver, brain, kidney, muscle and the like can also be adopted. For measurement of an in-vivo substance present in an in-vivo tissue, the laser beams are guided through an optical fiber to the portion to be measured of the in-vivo tissue. The measurement using an optical fiber may be applied, for example, to detection of an in-vivo substance in a tissue at a diseased portion (operated portion) during endoscopy, an abdominal operation or the like.

In the case where the measurement is conducted using an optical fiber, the optical path of the pump light and the Stokes light in the apparatus configuration shown in FIG. 2 is extended by use of the optical fiber, and irradiation of the sample S with the pump light and the Stokes light is carried out through an objective lens 3 mounted at the tip (distal end) of the optical fiber. In addition, the CARS light emitted from the sample S and returning to the objective lens 3 is detected as a spectrum. This ensures that the irradiation with the pump light and the Stokes light and the detection of the CARS spectrum can be conducted from the same direction in relation to the sample S. This technique is particularly useful in the case where the objective lens 3 is attached to the tip of an optical fiber and introduced into a living body so as to perform the measurement.

The in-vivo substance as the object of measurement in the present invention is not limited to HbAlc, and may widely include chemical substances present in living body tissues and cells. Specifically, examples of the chemical substances which can be adopted as the in-vivo substance include amino acids, peptides, proteins, nucleotide, nucleoside, nucleic acids, sugars, lipid, vitamins, hormones, metallic elements, and metallic element-containing proteins.

Furthermore, when an in-vivo substance of which the occurrence or accumulation in a living tissue is related to a specific disease or physiological function is adopted as the in-vivo substance in the present invention, it is possible to determine the disease condition or the physiological function. As a preferable example of such an in-vivo substance, the above-mentioned glycosylated hemoglobin (HbAlc) may be mentioned. The HbAlc and glycosylated proteins such as glycosylated albumin, glycosylated globulin and fructosamine are each used as a diagnostic marker which is an index to the hyperglycemic state in the diabetic pathologic conditions.

When the measuring methods according to the related art was applied to HbAlc, for example, it would be necessary to first collect blood from the patient, to separate and dissolve (lyse) the erythrocyte, to fractionate the hemoglobin by HPLC, and to measure the quantity of the fractionated HbAlc.

In contrast, when the method of measuring the quantity of an in-vivo substance according to an embodiment of the present invention is used, the quantity of HbAlc in the erythrocyte can be measured directly, without performing such an analysis using HPLC. Therefore, the patient's condition of blood sugar level can be grasped easily and swiftly. This ensures that evaluations of risk of crisis, prognosis, therapeutic results or the like can be performed speedily, without exerting physical burden on the patient.

Incidentally, when measurement of glycosylated albumin, glycosylated globulin, and/or fructosamine is conducted by the method according to an embodiment of the present invention, irradiation with the laser beams is conducted by focusing the laser beams on that location in a blood vessel V at which erythrocyte E is absent, as shown in FIG. 4B. In this case, it may be necessary to momentarily stop the bloodstream in the blood vessel V.

The method of measuring the quantity of an in-vivo substance according to an embodiment of the present invention can be utilized for detection of an in-vivo substance in a living tissue, in pharmacological tests and safety tests in the drug discovery field, for example. Besides, the method of measuring the quantity of an in-vivo substance according to an embodiment of the present invention can be used for evaluations of risk of crisis, prognosis, therapeutic results or the like.

The present application contains subject matter related to that disclosed in Japanese priority Patent Application JP 2008-166731 filed in the Japan Patent Office on Jun. 26, 2008, the entire content of which is hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A method of measuring the quantity of an in-vivo substance comprising the acts of:
irradiating an in-vivo substance with two near infrared femtosecond laser beams of different frequencies;
detecting coherent anti-Stokes Raman scattered light emitted from said in-vivo substance due to coincidence of a frequency difference between said two near infrared femtosecond laser beams with a natural frequency of said in-vivo substance; and
determining a quantity of said in-vivo substance, based on a peak intensity of a Raman scattering spectrum obtained.

2. The method according to claim 1, wherein said in-vivo substance is glycosylated hemoglobin.
